(19) United States
(12) Patent Application Publication
(10) Pub. No.: US 2026/0137360 A1
(43) Pub. Date: May 21, 2026

(54) METHOD, DEVICE, AND SYSTEM FOR X-RAY CT CONTRAST IMAGING USING MAGNETIC NANOPARTICLES

(71) Applicant: HUAZHONG UNIVERSITY OF SCIENCE AND TECHNOLOGY, Hubei (CN)

(72) Inventors: Xinchao Cui, Hubei (CN); Wenzhong Liu, Hubei (CN)

(73) Assignee: HUAZHONG UNIVERSITY OF SCIENCE AND TECHNOLOGY, Hubei (CN)

(21) Appl. No.: 19/181,302

(22) Filed: Apr. 16, 2025

(30) Foreign Application Priority Data
Jun. 13, 2024 (CN) 202410759779.9

(51) Int. Cl. A61B 6/00; A61B 6/03; A61B 6/04; A61B 6/42; A61K 49/04; G06T 12/30

(52) U.S. Cl. CPC A61B 6/481; A61B 6/032; A61B 6/0407; A61B 6/42; A61B 6/5205; A61K 49/04; G06T 12/30

(57) ABSTRACT

Disclosed are a method, device, and system for X-ray CT contrast imaging using magnetic nanoparticles. The method includes: rotating a target object containing magnetic nanoparticles as contrast agent for one circle relative to X-rays, recording multiple consecutive transmission projection images of the target object at all angles; for multiple transmission projection images at each angle, after conducting image post-processing operations, performing inverse projection transform on magnetic nanoparticle imaging projection images at all angles to obtain a tomographic imaging result. The image post-processing operation includes: time-frequency transform: converting the third dimension of the three-dimensional time domain matrix to frequency domain to obtain a three-dimensional frequency domain matrix; feature extraction: selecting a two-dimensional matrix with frequency of kf along the third dimension of the three-dimensional frequency domain image matrix; k is a positive integer; f is the frequency of the excitation magnetic field.

### CROSS-REFERENCE TO RELATED APPLICATION

This application claims the priority benefit of China application serial no. 202410759779.9, filed on Jun. 13, 2024. The entirety of the above-mentioned patent application is hereby incorporated by reference herein and made a part of this specification.

### BACKGROUND

### Field of the Disclosure

The present disclosure belongs to imaging technology using magnetic nanoparticles, more specifically, relates to a method, device and system for X-ray CT contrast imaging using magnetic nanoparticles.

### Description of Related Art

With the advancement of interventional radiology, angiography has evolved into a critical diagnostic methodology in clinical practice, particularly fulfilling an irreplaceable function in interventional therapeutics. Computed Tomography Angiography (CTA) constitutes a specialized interventional CT enhancement technique characterized by its expeditious implementation, non-invasive nature, and operational simplicity. Through appropriate post-processing protocols, CTA facilitates the lucid visualization of vascular details, thereby conferring significant diagnostic value in the identification of vascular anomalies, vascular pathologies, and the elucidation of relationships between lesions and vasculature.

Iodinated contrast agents are commonly utilized in CT angiography; however, individuals with iodine hypersensitivity may experience symptoms of iodine toxicity, which can severely impair renal function, thyroid function, and cause other common adverse reactions. Magnetic nanoparticles, as novel nanoscale contrast agents, have previously been employed in Magnetic Resonance Imaging (MRI) to enhance imaging contrast. Their non-toxic nature and amenability to surface modification enable surface-modified magnetic nanoparticles to precisely target lesional regions. Furthermore, quantitative detection of nanoparticle concentration distribution may be achieved based on their magnetic response, thereby facilitating preliminary diagnosis and targeted drug therapy. Additionally, the nanoparticles can serve as carriers to deliver therapeutic agents specifically to lesional regions, conferring significant importance in medical imaging, diagnosis, and treatment.

Furthermore, Computed Tomography (CT), which is electronic computer tomography, operates on the fundamental principle of utilizing the strong penetrative capability of X-rays and the differential absorption and transmission rates of X-rays by various substances to reflect the internal morphological structures of objects. Through image reconstruction methodologies, sectional imaging may be achieved. CT is categorized as structural imaging, generally employing CT values to represent substance density; specifically, a higher CT value corresponds to a higher substance density, thereby conferring significant capability for the deformation and localization of different tissues. However, during some specific periods, such as the precancerous stage, the disparity between pathological regions and normal structural tissues may be minimal, resulting in suboptimal efficacy when employing CT and other existing medical imaging modalities.

Magnetic nanoparticles possess superparamagnetic properties, enabling them to respond rapidly under magnetic field influence and exhibit zero magnetic hysteresis overall. Furthermore, the dynamic characteristics of magnetic nanoparticles under magnetic field influence alter their X-ray absorption rate. Additionally, the human body is relatively magnetically transparent for magnetic nanoparticles and does not change its X-ray absorption rate due to magnetic fields; therefore, utilizing magnetic nanoparticles as substitutes for iodine contrast agents in contrast imaging provides unique specificity and enhances imaging contrast. However, the responsive characteristics of magnetic nanoparticles under magnetic field influence are closely related to magnetic field frequency, whereas existing CT contrast imaging methods merely employ long exposure for simple image optimization processing and cannot effectively identify the imaging characteristics of magnetic nanoparticles, resulting in low imaging contrast.

### SUMMARY OF THE DISCLOSURE

In response to the deficiencies in existing technology and the need for improvement, this disclosure provides a method, device, and system for X-ray CT contrast imaging using magnetic nanoparticles. The purpose of the present disclosure is to utilize magnetic nanoparticles as contrast agents, and based on the responsive characteristics of magnetic nanoparticles to magnetic fields, to improve the processing method of captured X-ray transmission images, thereby effectively identifying the characteristics of magnetic nanoparticles and enhance imaging contrast.

To achieve the above purpose, according to one aspect of the present disclosure, a method for X-ray CT contrast imaging using magnetic nanoparticles is provided, including:
(S1) The target object in the imaging environment is rotated for one circle relative to the X-rays. During the rotation process, 1 consecutive transmission projection images of the target object at all angles are recorded, and transmission projection images are recorded at a total of t angles. Magnetic nanoparticles are employed as the contrast agent for the target object, and the imaging environment is formed by overlaying X-rays and a periodically changing excitation magnetic field parallel to the X-ray axial direction, wherein 1 and t are both positive integers greater than 1.
(S2) The 1 transmission projection images at all angles are established as a three-dimensional time domain matrix of M×N×1, then an image post-processing operation is conducted to obtain the magnetic nanoparticle contrast imaging projection image at the corresponding angle, wherein MXN represents the resolution of each transmission projection image.

The image post-processing operation includes:

Time-frequency transform: The third dimension of the three-dimensional time domain matrix is converted to frequency domain to obtain a three-dimensional frequency domain matrix.

Feature extraction: A two-dimensional matrix with frequency kf is selected along the third dimension of the three-dimensional frequency domain image matrix to obtain the magnetic nanoparticle contrast imaging projection image, wherein k is a positive integer, and f is the frequency of the excitation magnetic field.
(S3) Inverse projection transform is conducted on the magnetic nanoparticle contrast imaging projection images at t angles to obtain the X-ray CT contrast imaging result using magnetic nanoparticles.

Further, the image post-processing operation also includes:

Image segmentation: A three-dimensional time domain matrix is divided equally into sub-three-dimensional time domain matrices of m×n×1; wherein m<M, and n<N.

Image merging: Time-frequency transform and feature extraction are executed on each sub-three-dimensional time domain matrix respectively. After obtaining the magnetic nanoparticle contrast imaging projection image corresponding to each sub-three-dimensional time domain matrix, the magnetic nanoparticle contrast imaging projection images are connected in sequence to obtain the magnetic nanoparticle contrast imaging projection image at the corresponding angle.

Further, k=1 or 2.

According to another aspect of the present disclosure, a device for X-ray CT contrast imaging using magnetic nanoparticles is provided, including:

A control module is configured to control the target object in the imaging environment to rotate for one circle relative to the X-rays. Magnetic nanoparticles are employed as the contrast agent for the target object, and the imaging environment is formed by overlaying X-rays and a periodically changing excitation magnetic field parallel to the X-ray axial direction.

A detection module is configured to record 1 consecutive transmission projection images of the target object at all angles during the process of the target object rotating relative to the X-rays, and record transmission projection images at a total of t angles, wherein 1 is a positive integer greater than 1.

An image post-processing module is configured to establish the 1 transmission projection images at all angles as a three-dimensional time domain matrix of M×N×1, then conduct an image post-processing operation to obtain the magnetic nanoparticle contrast imaging projection image at the corresponding angle, wherein MXN represents the resolution of each transmission projection image.

And an inverse projection module is configured to conduct inverse projection transform on the magnetic nanoparticle contrast imaging projection images at t angles to obtain the X-ray CT contrast imaging result using magnetic nanoparticles.

Further, the image post-processing operation includes:

Time-frequency transform: The third dimension of the three-dimensional time domain matrix is converted to frequency domain to obtain a three-dimensional frequency domain matrix.

Feature extraction: A two-dimensional matrix with frequency kf is selected along the third dimension of the three-dimensional frequency domain image matrix to obtain the magnetic nanoparticle contrast imaging projection image, wherein k is a positive integer, and f is the frequency of the excitation magnetic field.

Further, the image post-processing operation also includes:

Image segmentation: A three-dimensional time domain matrix is divided equally into sub-three-dimensional time domain matrices of m×n×1; wherein m<M, and n<N.

Image merging: Time-frequency transform and feature extraction are executed on each sub-three-dimensional time domain matrix respectively. After obtaining the magnetic nanoparticle contrast imaging projection image corresponding to each sub-three-dimensional time domain matrix, the magnetic nanoparticle contrast imaging projection images are connected in sequence to obtain the magnetic nanoparticle contrast imaging projection image at the corresponding angle.

Further, k=1 or 2.

According to another aspect of the present disclosure, a system for X-ray CT contrast imaging using magnetic nanoparticles is provided, including: a transmission imaging device and the device for X-ray CT contrast imaging using magnetic nanoparticles provided by the present disclosure.

The transmission imaging device includes: a scan frame; an X-ray source; a detector, having a detection surface perpendicular to the X-ray axial direction along which the X-ray source is emitted, wherein the detector and the X-ray source are respectively disposed at both ends of the scan frame; Helmholtz coils, fixed at the middle part of the scan frame, and configured to provide a periodically changing excitation magnetic field parallel to the X-ray axial direction; and a scan bed, configured to carry the target object to be imaged, and during imaging, the scan bed is located between the two coils of the Helmholtz coils.

The X-ray source, the detector and the Helmholtz coils may rotate as a whole through the scan frame.

A control module is connected to the scan frame, and the detection module is connected to the detector.

Overall, through the technical solutions conceived by the present disclosure above, the following advantageous results may be obtained.
(1) The present disclosure, for target object injected with magnetic nanoparticles used as contrast agent, places the target object in an imaging environment formed by overlaying X-rays and the periodically changing excitation magnetic field parallel to the X-ray axial direction, and when the target object and the X-rays are at different angles, respectively obtains consecutive multiple transmission projection images, thus completing the acquisition of time domain images. For transmission projection images at all angles, after converting the acquired images from time domain to frequency domain through time-frequency transform, the images at frequency points (i.e., at the harmonic frequency of the periodically changing excitation magnetic field) where features of magnetic nanoparticle are more obvious are extracted as magnetic nanoparticle imaging projection images at the corresponding angle. Afterwards, by performing inverse projection transform on the magnetic nanoparticle imaging projection images at all angles, the X-ray CT contrast imaging result using magnetic nanoparticles may be obtained. The features of magnetic nanoparticles are accurately extracted in this result, and the imaging contrast is effectively improved.
(2) In a preferred embodiment of the present disclosure, before processing the time domain images at a specific angle, they will first be divided equally into multiple small areas. After executing time-frequency transform and feature extraction on each small area respectively, they are merged into magnetic nanoparticle imaging projection image at the corresponding angle. This may reduce the amount of data for time-frequency transform calculation, reduce the memory and time cost required for data processing, and improve imaging efficiency.
(3) In a preferred embodiment of the present disclosure, when extracting images with more obvious magnetic nanoparticle features from frequency domain images, specifically extracting images at the first harmonic or the second harmonic of the periodically changing excitation magnetic field, at these two frequency points, the response of magnetic nanoparticles is most obvious, and the signal strength is the highest. Extracting images at these points is beneficial for further improving imaging contrast.

### BRIEF DESCRIPTION OF THE DRAWINGS

FIG. 1 is a flowchart of a method for X-ray CT contrast imaging using magnetic nanoparticles provided by an exemplary embodiment of the present disclosure.

FIG. 2 is a schematic diagram of an image post-processing method provided by an exemplary embodiment of the present disclosure.

FIG. 3 is a schematic diagram of a transmission imaging device provided by an exemplary embodiment of the present disclosure.

In all drawings, the same reference numerals are used to indicate the same elements or structures, wherein: 1—scan bed, 2—X-ray source, 3—magnetic nanoparticle imaging area, 4—X-ray beam, 5—detector, 6—Helmholtz coil, 7—scan frame

### DESCRIPTION OF EMBODIMENTS

In order to make the purpose, technical solution and advantages of the present disclosure more comprehensible, the following will further describe the present disclosure in detail in conjunction with the drawings and embodiments. It should be understood that the specific embodiments described here are merely used to explain the present disclosure and not to limit the present disclosure. In addition, the technical features involved in various embodiments of the present disclosure described below may be combined with each other as long as they do not conflict with each other.

In the present disclosure, the terms “first”, “second”, etc. (if they exist) in the present disclosure and the drawings are used to distinguish similar objects, and are not necessarily used to describe a specific sequence or order.

Magnetic nanoparticles replace iodine reagents as contrast agents, which may solve the situation of patients' iodine allergies, and may improve imaging contrast. However, existing CT contrast imaging methods cannot effectively identify the features of magnetic nanoparticles from the detected image data when using magnetic nanoparticles as contrast agents, resulting in the inability to effectively utilize the advantages of magnetic nanoparticles as contrast agents, and the imaging contrast is not high. To address this problem, the present disclosure provides a method, device, and system for X-ray CT contrast imaging using magnetic nanoparticles. The overall approach consists in: based on the responsive characteristics of magnetic nanoparticles to magnetic fields, improving the processing mode of the captured X-ray transmission images to effectively identify the features of magnetic nanoparticles and improve imaging contrast.

The following are exemplary embodiments.

### Exemplary Embodiment 1

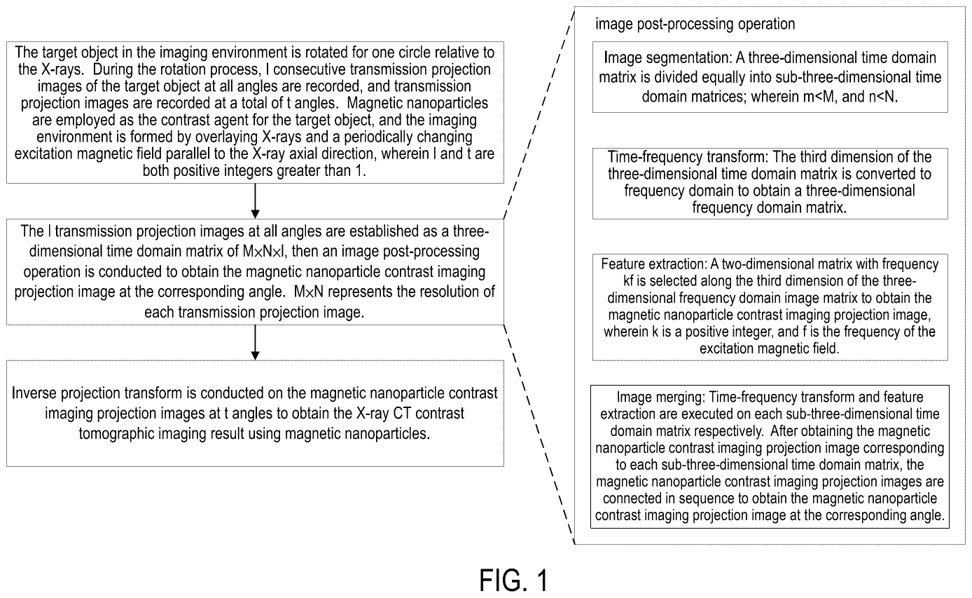

A method for X-ray CT contrast imaging using magnetic nanoparticles, as shown in FIG. 1, includes: steps (S1) to (S3).

As shown in FIG. 1, in this exemplary embodiment, step (S1) specifically includes: The target object in the imaging environment is rotated for one circle relative to the X-ray. During the rotation process, 1 consecutive transmission projection images of the target object at all angles are recorded.

Magnetic nanoparticles are employed as the contrast agent for the target object, and the imaging environment is formed by overlaying X-ray and a periodically changing excitation magnetic field parallel to the X-ray axial direction. Optionally, in this exemplary embodiment, the periodically changing excitation magnetic field is specifically a sinusoidal excitation magnetic field, which may be expressed as:

$$H ( t ) = {H}_{0} sin ( 2 π ft )$$

Wherein, H₀ is the amplitude of the sinusoidal excitation magnetic field, and f is the frequency of the sinusoidal excitation magnetic field.

Since the excitation magnetic field will cause the magnetic nanoparticles to respond, resulting in changes in the X-ray absorption rate of the magnetic nanoparticles, before and after applying the alternating magnetic field, the transmission projection images detected by the detector will change.

Optionally, in this exemplary embodiment, transmission projection images are recorded at 360 angles at intervals of 1°.

Optionally, in this exemplary embodiment, at each angle, an image is acquired for Tₛ seconds using a detector with a frame rate of Fₛ, so as to obtain 1 consecutive transmission projection images at that angle, which are then recorded. Since this exemplary embodiment uses magnetic nanoparticles as contrast agents, which may effectively enhance imaging contrast, the efficiency of capturing a single transmission projection image may be significantly improved, making it possible to capture multiple transmission projection images at each angle.

With M×N representing the resolution of the transmission projection image, each transmission projection image represents the distribution of transmission signal intensity in the M×N imaging area at the corresponding moment, and 1 transmission projection images constitute the time domain image acquisition result at that angle; 1 is a positive integer greater than 1, in this exemplary embodiment, 1=Fₛ×Tₛ.

Based on step (S1), this exemplary embodiment completes the time domain image acquisition at various angles.

As shown in FIG. 1, in this exemplary embodiment, step (S2) specifically includes: The 1 transmission projection images at all angles are established as a three-dimensional time domain matrix of M×N×1, then an image post-processing operation is conducted to obtain the magnetic nanoparticle contrast imaging projection image at the corresponding angle.

Each transmission projection image records the transmission signal intensity at M×N pixel positions, and each transmission projection image may be viewed as a two-dimensional matrix. By arranging the transmission projection images according to the recording time sequence, a three-dimensional time domain matrix of M×N×1 may be obtained.

In the exemplary embodiment, the image post-processing operation includes:

Image segmentation: A three-dimensional time domain matrix is divided equally into sub-three-dimensional time domain matrices of m×n×1; wherein m<M, and n<N.

Time-frequency transform: The third dimension of the three-dimensional time domain matrix is converted to frequency domain to obtain a three-dimensional frequency domain matrix.

Feature extraction: A two-dimensional matrix with frequency kf is selected along the third dimension of the three-dimensional frequency domain image matrix to obtain the magnetic nanoparticle contrast imaging projection image, wherein k is a positive integer, and f is the frequency of the excitation magnetic field.

Image merging: Time-frequency transform and feature extraction are executed on each sub-three-dimensional time domain matrix respectively. After obtaining the magnetic nanoparticle contrast imaging projection image corresponding to each sub-three-dimensional time domain matrix, the magnetic nanoparticle contrast imaging projection images are connected in sequence to obtain the magnetic nanoparticle contrast imaging projection image at the corresponding angle.

Under the effect of the magnetic field, at the harmonic frequency of the periodically changing excitation magnetic field, i.e., at the frequency points with frequency kf, the responsive features of magnetic nanoparticles are more obvious, this exemplary embodiment first converts the image from time domain to the frequency domain, then extracts the image at the frequency point (i.e., at the harmonic frequency of the periodically changing excitation magnetic field) where features of magnetic nanoparticle are more obvious as magnetic nanoparticle imaging projection images at the corresponding angle, which may accurately capture the features of magnetic nanoparticles in the final imaging result. This exemplary embodiment further discovers that the images at the first harmonic or second harmonic of the periodically changing excitation magnetic field, at these two frequency points, the response of magnetic nanoparticles is most obvious, with the strongest signal intensity. Extracting the image at this point is conducive to further improving the imaging contrast. Therefore, as a preferred implementation mode, this exemplary embodiment specifically extracts the image at f during feature extraction.

Through image segmentation, the amount of data for time-frequency transform calculation may be effectively reduced, decreasing the memory and time costs required for data processing, and improving imaging efficiency.

Optionally, in this exemplary embodiment, the time-frequency transform may be completed through Fast Fourier Transform (FFT).

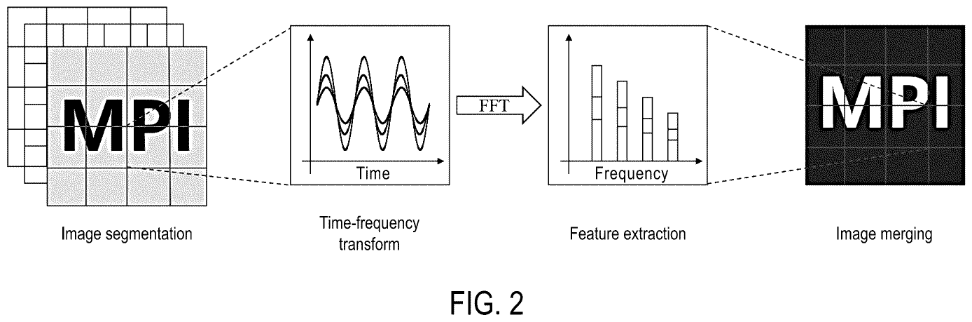

As shown in FIG. 2, it is a schematic diagram of the above image post-processing operations performed on the transmission projection image acquired at a specific angle.

Based on step (S2), this exemplary embodiment obtains the magnetic nanoparticle imaging projection images at various angles from the original time domain image acquisition results at various angles.

As shown in FIG. 1, in this exemplary embodiment, step (S3) specifically includes: Inverse projection transform is conducted on the magnetic nanoparticle contrast imaging projection images at all angles to obtain the X-ray CT contrast imaging result using magnetic nanoparticles.

It is easy to understand that, with t representing the total number of angles, for all magnetic nanoparticle imaging projection images at t angles, two modes may be used for inverse projection transform. One is to use a three-dimensional matrix of M×N×t for inverse projection, which makes it possible to obtain an X-ray CT contrast imaging result using magnetic nanoparticles with M×M size for N tomographic sections. The other is to use a three-dimensional matrix of N×M×t for inverse projection, which makes it possible to obtain an X-ray CT contrast imaging result using magnetic nanoparticles with N×N size for M tomographic sections. In practical applications, the inverse projection transform mode may be flexibly selected. Optionally, in this exemplary embodiment, the former mode is used for inverse projection transform. Therefore, after inverse projection transform, this exemplary embodiment will obtain an X-ray CT contrast imaging result using magnetic nanoparticles with M×M size for N tomographic sections.

In general, the embodiment, for target object injected with magnetic nanoparticles used as contrast agent, places the target object in an imaging environment formed by overlaying X-rays and the periodically changing excitation magnetic field parallel to the X-ray axial direction, and when the target object and the X-rays are at different angles, respectively obtains consecutive multiple transmission projection images, thus completing the acquisition of time domain images. For transmission projection images at all angles, after converting the acquired images from time domain to frequency domain through time-frequency transform, the images at frequency points (i.e., at the harmonic frequency of the periodically changing excitation magnetic field) where features of magnetic nanoparticle are more obvious are extracted as magnetic nanoparticle imaging projection images at the corresponding angle. Afterwards, by performing inverse projection transform on the magnetic nanoparticle imaging projection images at all angles, the X-ray CT contrast imaging result using magnetic nanoparticles may be obtained. The features of magnetic nanoparticles are accurately extracted in this result, and the imaging contrast is effectively improved.

### Exemplary Embodiment 2

A method for X-ray CT contrast imaging using magnetic nanoparticles. This exemplary embodiment is similar to the above Exemplary Embodiment 1, with the difference being that in this exemplary embodiment, after obtaining 1 consecutive transmission projection images at various angles, during image post-processing operation, no image segmentation is performed. Instead, time-frequency transform and feature extraction are executed on the entire three-dimensional time domain matrix. The extracted image is the magnetic nanoparticle imaging projection image at the corresponding angle.

### Exemplary Embodiment 3

A device for X-ray CT contrast imaging using magnetic nanoparticles, including:

A control module is configured to control the target object in the imaging environment to rotate for one circle relative to the X-rays. Magnetic nanoparticles are employed as the contrast agent for the target object, and the imaging environment is formed by overlaying X-rays and a periodically changing excitation magnetic field parallel to the X-ray axial direction.

A detection module is configured to record 1 consecutive transmission projection images of the target object at all angles during the process of the target object rotating relative to the X-rays, and record transmission projection images at a total of t angles, wherein 1 and t are both positive integers greater than 1.

An image post-processing module is configured to establish the 1 transmission projection images at all angles as a three-dimensional time domain matrix of M×N×1, then conduct an image post-processing operation to obtain the magnetic nanoparticle contrast imaging projection image at the corresponding angle, wherein M×N represents the resolution of each transmission projection image.

And an inverse projection module is configured to conduct inverse projection transform on the magnetic nanoparticle contrast imaging projection images at t angles to obtain the X-ray CT contrast imaging result using magnetic nanoparticles.

The image post-processing operation includes:

Image segmentation: A three-dimensional time domain matrix is divided equally into sub-three-dimensional time domain matrices of m×n×1; wherein m<M, and n<N.

Time-frequency transform: The third dimension of the three-dimensional time domain matrix is converted to frequency domain to obtain a three-dimensional frequency domain matrix.

Feature extraction: A two-dimensional matrix with frequency kf is selected along the third dimension of the three-dimensional frequency domain image matrix to obtain the magnetic nanoparticle contrast imaging projection image, wherein k is a positive integer, and f is the frequency of the excitation magnetic field.

Image merging: Time-frequency transform and feature extraction are executed on each sub-three-dimensional time domain matrix respectively. After obtaining the magnetic nanoparticle contrast imaging projection image corresponding to each sub-three-dimensional time domain matrix, the magnetic nanoparticle contrast imaging projection images are connected in sequence to obtain the magnetic nanoparticle contrast imaging projection image at the corresponding angle.

In this exemplary embodiment, the specific implementation modes of each module may refer to the description in the above Exemplary Embodiment 1, which will not be repeated here.

### Exemplary Embodiment 4

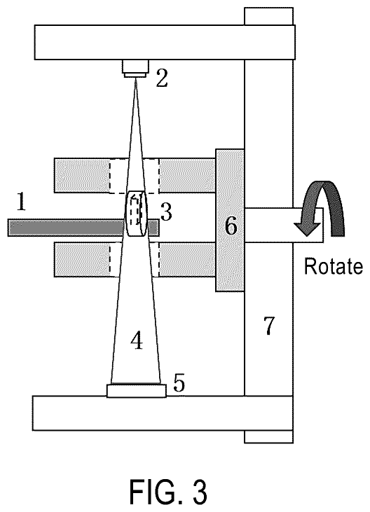

A system for X-ray CT contrast imaging using magnetic nanoparticles, as shown in FIG. 3, includes: a transmission imaging device and the device for X-ray CT contrast imaging using magnetic nanoparticles provided by the present disclosure.

The transmission imaging device includes: a scan frame 7; an X-ray source 2; a detector 5, having a detection surface perpendicular to the X-ray axial direction along which the X-ray source 2 is emitted, wherein the detector 5 and the X-ray source 2 are respectively disposed at both ends of the scan frame 7; Helmholtz coils 6, fixed at the middle part of the scan frame 7, and configured to provide a periodically changing excitation magnetic field parallel to the X-ray axial direction; and a scan bed 1, configured to carry the target object to be imaged, and during imaging, the scan bed 1 is located between the two coils of the Helmholtz coils 6.

The X-ray source 2, the detector 5 and the Helmholtz coils 6 may rotate as a whole through the scan frame 7.

The control module is connected to the scan frame 7, and the detection module is connected to the detector 5.

The Helmholtz coil is a device for producing a uniform magnetic field in a small area, usually consisting of two coils. Due to the open nature of the Helmholtz coil, it is easy to place the Helmholtz coil or remove the Helmholtz coil from other instruments, and visual observation of the Helmholtz coil may also be directly made. This exemplary embodiment uses the Helmholtz coil to provide a periodically changing excitation magnetic field, allowing the target object to conveniently enter and exit the imaging area.

In the system for X-ray CT contrast imaging using magnetic nanoparticles provided by this exemplary embodiment, during operation, the target object containing magnetic nanoparticle used as the contrast agent may be moved to the imaging area 3 through the scan bed 1, with the X-ray source 2 providing the X-ray beam 4 to the imaging area 3, and the Helmholtz coils 6 providing a periodically changing excitation magnetic field parallel to the axial direction of the X-ray beam 4. By sending corresponding instructions to the scan frame 7 through the control module, the scan frame 7 together with the X-ray source 2, the detector 5 and the Helmholtz coils 6 may rotate circumferentially as a whole relative to the target object. At each angle, the transmission projection image is detected by the detector and sent to the detection module for recording. After performing image post-processing and inverse projection on images at all angles by the image post-processing module and inverse projection module, the corresponding X-ray CT contrast imaging result using magnetic nanoparticles is obtained.

Those skilled in the art will readily understand that the above description is only a preferred exemplary implementation of the present disclosure and is not intended to limit the present disclosure. Any modifications, equivalent replacements, and improvements made within the spirit and principles of the present disclosure should be included within the scope to be protected by the present disclosure.

What is claimed is:

1. A method for X-ray CT contrast imaging using magnetic nanoparticles, comprising:
(S1) a target object in an imaging environment is rotated for one circle relative to X-rays, during a rotation process, 1 consecutive transmission projection images of the target object at all angles are recorded, and the transmission projection images are recorded at a total of t angles, wherein the magnetic nanoparticles are employed as a contrast agent for the target object, and the imaging environment is formed by overlaying the X-rays and a periodically changing excitation magnetic field parallel to an X-ray axial direction, wherein 1 and t are both positive integers greater than 1;
(S2) the 1 transmission projection images at the all angles are established as a three-dimensional time domain matrix of M×N×1, then an image post-processing operation is conducted to obtain a magnetic nanoparticle contrast imaging projection image at a corresponding angle, wherein M×N represents a resolution of each of the transmission projection images,
the image post-processing operation comprises:
time-frequency transform: a third dimension of the three-dimensional time domain matrix is converted to a frequency domain to obtain a three-dimensional frequency domain matrix;
feature extraction: a two-dimensional matrix with a frequency kf is selected along a third dimension of the three-dimensional frequency domain image matrix to obtain the magnetic nanoparticle contrast imaging projection image, wherein k is a positive integer, and f is a frequency of the excitation magnetic field;
(S3) inverse projection transform is conducted on the magnetic nanoparticle contrast imaging projection images at the t angles to obtain an X-ray CT contrast imaging result using the magnetic nanoparticles.

2. The method for X-ray CT contrast imaging using the magnetic nanoparticles according to claim 1, wherein the image post-processing operation further comprises:
image segmentation: the three-dimensional time domain matrix is divided equally into sub-three-dimensional time domain matrices of m×n×1, wherein m<M, and n<N;
image merging: time-frequency transform and feature extraction are executed on each of the sub-three-dimensional time domain matrices respectively, after obtaining the magnetic nanoparticle contrast imaging projection image corresponding to each of the sub-three-dimensional time domain matrices, the magnetic nanoparticle contrast imaging projection images are connected in sequence to obtain the magnetic nanoparticle contrast imaging projection image at the corresponding angle.

3. The method for X-ray CT contrast imaging using the magnetic nanoparticles according to claim 1, wherein k=1 or 2.

4. The method for X-ray CT contrast imaging using the magnetic nanoparticles according to claim 2, wherein k=1 or 2.

5. A device for X-ray CT contrast imaging using magnetic nanoparticles, comprising:
a control module, configured to control a target object in an imaging environment to rotate for one circle relative to X-rays, wherein the magnetic nanoparticles are employed as a contrast agent for the target object, and the imaging environment is formed by overlaying the X-rays and a periodically changing excitation magnetic field parallel to an X-ray axial direction;
a detection module, configured to record 1 consecutive transmission projection images of the target object at all angles during the process of the target object rotating relative to the X-rays, and record the transmission projection images at a total of t angles, wherein 1 and t are both positive integers greater than 1;
an image post-processing module, configured to establish the 1 transmission projection images at the all angles as a three-dimensional time domain matrix of M×N×1, then conduct an image post-processing operation to obtain a magnetic nanoparticle contrast imaging projection image at a corresponding angle, wherein M×N represents a resolution of each of the transmission projection images; and
an inverse projection module, configured to conduct inverse projection transform on the magnetic nanoparticle contrast imaging projection images at the t angles to obtain an X-ray CT contrast imaging result using the magnetic nanoparticles,
wherein the image post-processing operation comprises:
time-frequency transform: a third dimension of the three-dimensional time domain matrix is converted to a frequency domain to obtain a three-dimensional frequency domain matrix;
feature extraction: a two-dimensional matrix with a frequency kf is selected along a third dimension of the three-dimensional frequency domain image matrix to obtain the magnetic nanoparticle contrast imaging projection image, wherein k is a positive integer, and f is a frequency of the excitation magnetic field.

6. The device for X-ray CT contrast imaging using the magnetic nanoparticles according to claim 5, wherein the image post-processing operation further comprises:
image segmentation: the three-dimensional time domain matrix is divided equally into sub-three-dimensional time domain matrices of m×n×1, wherein m<M, and n<N;
image merging: time-frequency transform and feature extraction are executed on each of the sub-three-dimensional time domain matrices respectively, after obtaining the magnetic nanoparticle contrast imaging projection image corresponding to each of the sub-three-dimensional time domain matrices, the magnetic nanoparticle contrast imaging projection images are connected in sequence to obtain the magnetic nanoparticle contrast imaging projection image at the corresponding angle.

7. The device for X-ray CT contrast imaging using the magnetic nanoparticles according to claim 5, wherein k=1 or 2.

8. The device for X-ray CT contrast imaging using the magnetic nanoparticles according to claim 6, wherein k=1 or 2.

9. A system for X-ray CT contrast imaging using magnetic nanoparticles, comprising:
a transmission imaging device and the device for X-ray CT contrast imaging using the magnetic nanoparticles according to claim 5,
wherein the transmission imaging device comprises:
a scan frame;
an X-ray source;
a detector, having a detection surface perpendicular to the X-ray axial direction along which the X-ray source is emitted, wherein the detector and the X-ray source are respectively disposed at both ends of the scan frame;
Helmholtz coils, fixed at a middle part of the scan frame, and configured to provide a periodically changing excitation magnetic field parallel to the X-ray axial direction; and
a scan bed, configured to carry the target object to be imaged, wherein during imaging, the scan bed is located between two coils of the Helmholtz coils,
wherein the X-ray source, the detector and the Helmholtz coils are able to rotate as a whole through the scan frame, and
the control module is connected to the scan frame, and the detection module is connected to the detector.

10. A system for X-ray CT contrast imaging using magnetic nanoparticles, comprising:
a transmission imaging device and the device for X-ray CT contrast imaging using the magnetic nanoparticles according to claim 6,
wherein the transmission imaging device comprises:
a scan frame;
an X-ray source;
a detector, having a detection surface perpendicular to the X-ray axial direction along which the X-ray source is emitted, wherein the detector and the X-ray source are respectively disposed at both ends of the scan frame;
Helmholtz coils, fixed at a middle part of the scan frame, and configured to provide a periodically changing excitation magnetic field parallel to the X-ray axial direction; and
a scan bed, configured to carry the target object to be imaged, wherein during imaging, the scan bed is located between two coils of the Helmholtz coils,
wherein the X-ray source, the detector and the Helmholtz coils are able to rotate as a whole through the scan frame, and
the control module is connected to the scan frame, and the detection module is connected to the detector.

11. A system for X-ray CT contrast imaging using magnetic nanoparticles, comprising:
a transmission imaging device and the device for X-ray CT contrast imaging using the magnetic nanoparticles according to claim 7,
wherein the transmission imaging device comprises:
a scan frame;
an X-ray source;
a detector, having a detection surface perpendicular to the X-ray axial direction along which the X-ray source is emitted, wherein the detector and the X-ray source are respectively disposed at both ends of the scan frame;
Helmholtz coils, fixed at a middle part of the scan frame, and configured to provide a periodically changing excitation magnetic field parallel to the X-ray axial direction; and
a scan bed, configured to carry the target object to be imaged, wherein during imaging, the scan bed is located between two coils of the Helmholtz coils,
wherein the X-ray source, the detector and the Helmholtz coils are able to rotate as a whole through the scan frame, and
the control module is connected to the scan frame, and the detection module is connected to the detector.
